(19) United States
(12) Patent Application Publication
(10) Pub. No.: US 2011/0300209 A1
(43) Pub. Date: Dec. 8, 2011

(54) MODIFIED RELEASE SOLID PHARMACEUTICAL COMPOSITIONS OF TRIMETAZIDINE AND PROCESS THEREOF

(75) Inventors: Pradeep G. Surve, Mumbai (IN); Pankaj S. Mandpe, Mumbai (IN); Unmesh H. Chavan, Mumbai (IN); Jaideep T. Patil, Pune (IN); Animesh S. Salunkhe, Karad (IN)

(73) Assignee: MICRO LABS LIMITED, Bangalore (IN)

(21) Appl. No.: 13/145,237

(22) PCT Filed: Jan. 18, 2010
(86) PCT No.: PCT/IB10/00075

§ 371 Date: Aug. 16, 2011

(30) Foreign Application Priority Data
Jan. 20, 2009 (IN) 124/MUM/2009

(51) Int. Cl. A61K 9/52; A61K 9/22; A61P 9/10; A61K 9/48; A61P 3/00; A61K 31/495; A61K 9/14

(52) U.S. Cl. 424/452; 514/252.12; 424/457; 424/468; 424/490; 424/465; 424/497

(57) ABSTRACT

There is provided a modified release solid pharmaceutical composition comprising Trimetazidine and polyethylene oxide, wherein the composition does not include any lubricant.

### FIELD OF THE INVENTION

There is provided a modified release solid pharmaceutical composition comprising Trimetazidine and polyethylene oxide, wherein the composition does not include any lubricant.

### BACKGROUND OF THE INVENTION

Trimetazidine is a metabolic modulator which has demonstrated anti-ischemic effects in patients with angina. Unlike the conventional classes of antianginal drugs the efficacy of trimetazidine is not dependent on reduction in the heart rate or blood pressure.

The efficacy and safety of trimetazidine in the treatment of patients with coronary heart disease and stable angina has been demonstrated in a number of randomized, controlled clinical trials.

Trimetazidine is administered orally in doses of 40 to 60 mg daily in divided doses as immediate release preparations; usually in practice 20 mg preparation is given twice or thrice daily to ensure relatively constant plasma levels. In clinical practice, 35 mg. tablets are also often prescribed twice a day. However as the drug is absorbed quickly, immediate release forms tend to give much higher levels immediately after administration and a low level at the time of next dose.

Modified release compositions of trimetazidine have been developed to provide relatively constant plasma levels and sustained antianginal and anti ischemic efficacy round the clock. The aim of the modified release compositions is to achieve minimum therapeutic concentrations of drug in the plasma, maintain steady concentration without much fluctuation and increase duration of concentration plateau. It also helps in patient compliance.

EP 1195160 B1 discloses a sustained release matrix pharmaceutical composition containing active ingredient trimetazidine with hydrocolloid forming materials and/or hydrophobic polymers and/or other hydrophobic materials as a retardant.

EP 0673649 B1 discloses a prolonged release of trimetazidine which is controlled by using a reservoir system in which a mixture of polymer that is insoluble in water and a plasticizer is in the form of a film that coats tablets or mini granules comprising trimetazidine.

EP 1104673 A1 discloses a galenic disintegrating agent free pharmaceutical composition of an active principle, notably of an active principle having anti-ischemic action characterized in that it comprises at least one diluting agent comprising a hydrogen carbonate.

U.S. Pat. No. 4,814,176 discloses sustained release preparation comprising chitin, chitosan or a mixture of thereof, anionic polymers having carboxyl group or a group capable of providing the same and a pharmaceutical active agent including trimetazidine hydrochloride.

EP 1108424B1 discloses matrix tablet for the prolonged release of trimetazidine characterized in that the prolonged release is controlled by the use of a cellulose derivative polymer present in the matrix selected from hydroxy propyl cellulose, hydroxy ethyl cellulose, hydroxy methyl cellulose, methyl cellulose and hydroxy propyl methyl cellulose.

It is known in the prior art that various grades of hydroxy propyl methyl cellulose are used in the modified release compositions. The HPMC matrix based system work by the swell and gel technique i.e. they swell by taking in fluids and further gel to provide a matrix which provides sustained effect facilitated by diffusion of the drug. However it is seen that higher viscosity grade HPMC release the drug slowly in the initial phases and the release rate is increased as the time progresses. Exactly the opposite in release is observed when one uses the low viscosity grade HPMC, where in the release is fast in the beginning and slows down as the time progresses.

FR 2885807 B1 discloses a sustained release solid pharmaceutical composition comprising trimetazidine or the pharmaceutically acceptable salt thereof combined with at least one type of polyethylene oxide, at least one type of lubricating agent. It discloses various lubricants including magnesium and calcium stearates.

Polyethylene oxides are among various hydrophilic polymers that, in the presence of water, control the release of the active ingredient. The primary disadvantage of the use of magnesium and calcium stearate with polyethylene oxide lies in the fact that magnesium and calcium stearate are extremely hydrophobic. This hydrophobicity hinders dissolution and disintegration time of composition containing polyethylene oxide. Another factor which acts to hinder dissolution and disintegration time of pharmaceutical composition containing magnesium or calcium stearate is their electrostatic attraction with polyethylene oxide.

The inventors have now developed a modified release solid pharmaceutical composition comprising Trimetazidine and polyethylene oxide, wherein the composition does not include any lubricant. Thus, the disadvantage caused because of the presence of lubricant is eliminated.

### SUMMARY OF THE INVENTION

In one general aspect, there is provided a modified release solid pharmaceutical composition comprising Trimetazidine and polyethylene oxide, wherein the composition does not include any lubricant.

Embodiments of the pharmaceutical composition may include one or more of the following features. The solid pharmaceutical composition may be in the form of one or more of tablet, capsule, powder, disc, caplet, granules, pellets, granules in capsule, minitablets, minitablets in capsule, pellets in capsule, sachet and the like. The solid pharmaceutical composition also includes multilayer tablets.

In one of the embodiments, the solid pharmaceutical composition is in the form of matrix type system, a reservoir type dosage form, multiple unit composition or combinations of one or more.

In another embodiment, the solid pharmaceutical composition as described herein may include one or more pharmaceutically acceptable inert excipients. The pharmaceutically acceptable inert excipients as used herein include binders, fillers, disintegrants, and the like.

In yet another embodiment the solid pharmaceutical composition as described herein may be coated.

In yet another embodiment, the coating may be functional or non-functional coating. The functional coating includes a release modifying polymer.

In another embodiment, the release modifying polymer includes combination of water soluble polymer and water insoluble polymer.

In another embodiment, the coating further comprises pharmaceutically acceptable inert excipients. The pharmaceutically acceptable inert excipients may include one or more of film formers, solvents, opacifiers, colorants and the like.

In one of the embodiments, the coating as described herein includes one or more pharmaceutically acceptable inert excipients. The pharmaceutically acceptable inert excipients as used herein include film formers, solvents, colorants and the like.

In another aspect, there is provided a process for the preparation of a modified release solid pharmaceutical composition comprising Trimetazidine and polyethylene oxide, wherein the composition does not include any lubricant, the process comprising the steps of: (a) granulating Trimetazidine optionally with pharmaceutically acceptable inert excipients to form granules; (b) drying the granules; (c) mixing the dried granules with polyethylene oxide to form a blend; and (d) compressing the blend into suitable sized tablet; (e) optionally coating the tablet.

The details of one or more embodiments of the invention are set forth in the description below. Other features, objects, and advantages of the invention will be apparent from the detailed description and claims.

### DETAILED DESCRIPTION OF THE INVENTION

The term “modified release” is defined for purposes of the invention as a method of drug delivery where the rate of release of the trimetazidine from the composition is not solely dependent on the concentration of trimetazidine remaining in the composition and/or the solubility of the trimetazidine in the medium surrounding the composition, and where the time course and/or location of release of trimetazidine from a pharmaceutical composition are chosen to accomplish therapeutic or convenience objectives not offered by conventional dosage forms.

“Trimetazidine” as used herein includes all its polymorphic forms and the pharmaceutically acceptable salts thereof, including dihydrochloride salts.

The solid pharmaceutical composition comprises from 2 to 50 weight % of Trimetazidine.

The term “solid pharmaceutical composition” may include one or more of tablet, capsule, powder, disc, caplet, granules, pellets, granules in capsule, minitablets, minitablets in capsule, pellets in capsule, sachet and the like. The solid pharmaceutical composition also includes multilayer tablets. The solid pharmaceutical compositions are meant for oral administration.

The term “tablet” includes pharmaceutical compositions of all shapes and sizes, whether coated or uncoated.

Polyethylene oxides are among various hydrophilic polymers that, in the presence of water, control the release of the active ingredient. Polyethylene oxide as used herein may include one or more grades with different viscosities in the range of 5,000 to 10,000 in 1% aqueous solution. The commercially available grades of polyethylene oxide available as “Polyox” may be used. The concentration of polyethylene oxide is within 30% to 60% by weight of composition.

The solid pharmaceutical composition may include one or more pharmaceutically acceptable inert excipients. The pharmaceutically acceptable inert excipients as used herein include binders, fillers, disintegrants, and the like.

Suitable binders may include one or more of povidone, starch, gums, hydroxypropylmethyl cellulose, and the like.

Suitable fillers may include one or more of microcrystalline cellulose, calcium phosphate, calcium sulfate, kaolin, starch, powdered sugar, and the like.

Suitable disintegrants may include one or more of starch, croscarmellose sodium, crospovidone, sodium starch glycolate, and the like.

The solid pharmaceutical composition is in the form of matrix type system, a reservoir type dosage form, multiple unit dosage form or combinations of one or more dosage forms.

Matrix type compositions are those in which the drug is distributed uniformly in polyethylene oxide and reservoir type compositions utilize polymeric coatings over the core of Trimetazidine. The core may be in the form of granule, tablet, pellet, capsule and the like.

The solid pharmaceutical composition as described herein may be coated. The coating may be functional or non-functional coating.

The coating may further comprise pharmaceutically acceptable inert excipients. The pharmaceutically acceptable inert excipients in the coating may include one or more of film formers, solvents, opacifiers, colorants and the like. The coating does not include any plasticizer.

Suitable film formers may include one or more of hydroxypropyl methyl cellulose, methyl hydroxyethyl cellulose, ethyl cellulose, hydroxypropyl cellulose, povidone, sodium carboxymethyl cellulose, acrylates and the like.

Suitable solvents may include one or more of water, ethanol, methanol, isopropanol, chloroform, acetone, methylethyl ketone, methylene chloride and the like.

Suitable opacifiers include titanium oxide, talc, aluminium silicate, magnesium carbonate, calcium sulfate and the like.

Suitable colorants include iron oxide, ferric oxide yellow, lake of Tartrazine, allura red, lake of quinoline yellow, lake of erythrosine and the like.

The functional coating includes a release modifying polymer. The release modifying polymer is one or more of water soluble polymer or water insoluble polymer.

The water soluble polymer may include one or more of hydroxypropyl methylcellulose, hydroxypropyl cellulose and the like.

The water insoluble polymer may include one or more of ethyl acrylate, methyl methacrylate and ethyl trimethylammonium chloride methacrylate, ethyl cellulose and the like.

The combination of water soluble and insoluble polymer may be used. The ratio of water soluble to water insoluble polymer is determined by the particular combination of polymers selected. The ratio of water soluble polymer to water insoluble polymer varies from 1:5 to 5:1. The water soluble and insoluble polymer may be hydroxypropyl methyl cellulose and ethyl cellulose respectively.

The coating may be applied as solution/dispersion of coating ingredients using any conventional techniques known in the prior art such as spray coating in a conventional coating pan or fluidized bed processor or dip coating.

There is provided a process for the preparation of a modified release solid pharmaceutical composition comprising Trimetazidine and polyethylene oxide, wherein the composition does not include any lubricant, the process comprising the steps of:

(a) granulating Trimetazidine optionally with pharmaceutically acceptable inert excipients to form granules;
(b) drying the granules;
(c) mixing the dried granules with polyethylene oxide to form a blend; and
(d) compressing the blend into suitable sized tablet;
(e) optionally coating the tablet.

When the solid pharmaceutical composition is in the form of capsule, the tablet may be filled into capsule. The granules blended with polyethylene oxide may be filled into the capsule. The capsule may further be optionally film coated with functional or non-functional coating.

The process for preparing the solid pharmaceutical composition may be wet granulation process. The granulation may be carried out by aqueous or non-aqueous method. The non-aqueous method uses non-aqueous solvents.

The solvents used for wet granulation process include one or more of water, ethanol, methanol, propanol, isopropanol, acetone, methylene chloride, ethyl acetate.

The terms “comprise”, “comprising, “include”, “including” are intended to be open, non-limiting, unless contrary is indicated.

The present invention is further illustrated below by reference to the following example. However, it will be apparent to those skilled in the art that various modifications and variations can be made in the methods and compositions of the present invention without departing from the scope of the invention. Thus, the present invention covers the modifications and variations of this invention provided they come within the scope of the claims.

### Examples 1 and 2

TABLE 1

| | | Mg/tablet | |
|---|---|---|---|
| SN | Ingredients | Example 1 | Example 2 |
| 1 | Trimetazidine | 35.000 | 35.000 |
| 2 | Microcrystalline cellulose | 55.000 | 53.000 |
| 3 | Polyethylene oxide | 80.000 | 90.000 |
| | (Polyox WSR 303 LEO) | | |
| 4 | Purified water | q.s. | q.s. |

Coating

| | | | |
|---|---|---|---|
| 5 | Hydroxypropyl methylcellulose | 11.345 | 15.525 |
| 6 | Ethyl cellulose | 5.463 | 7.475 |
| 7 | Titanium dioxide | 2.170 | 2.970 |
| 8 | Red iron oxide | 0.022 | 0.030 |
| 9 | Isopropyl alcohol | q.s. | q.s. |
| 10 | Methylene chloride | q.s. | q.s. |

| | | |
|---|---|---|
| Final weight of the tablet | 189.000 | 204.000 |

### Procedure

1. Trimetazidine was blended with microcrystalline cellulose to form a mixture.
2. The mixture in step 1 was granulated with purified water to form granules.
3. The granules were dried and milled.
4. The milled granules were mixed with polyethylene oxide to form a blend.
5. The blend was compressed into suitable sized tablets.
6. Hydroxypropyl methylcellulose, ethyl cellulose, titanium dioxide and Red iron oxide were suspended in isopropyl alcohol and methylene chloride to form a coating suspension.
7. The tablets in step 5 were coated with coating suspension formed in step 6.

TABLE 2
In-vitro release pattern of modified release pharmaceutical
composition comprising Trimetazidine as per Example 1 and 2 in
USP I apparatus in 900 ml of phosphate buffer 6.8 pH at
100 RPM at a temperature of 37° C. ± 0.5° C.

| | % Trimetazidine released | |
|---|---|---|
| Time (hr) | Example 1 | Example 2 |
| 0 | 0.00 | 0 |
| 1 | 32.4 | 35.9 |
| 2 | 60.3 | 59.4 |
| 3 | 76.6 | 74.6 |
| 4 | 85.7 | 83.6 |
| 5 | 92.1 | 89.5 |
| 6 | 95.0 | 92.9 |
| 7 | 96.6 | 93.9 |
| 8 | 98.6 | 96.2 |
| 9 | 100.1 | 96.9 |
| 10 | 101.0 | 98.5 |
| 11 | 104.7 | 98.2 |
| 12 | 101.8 | 99.7 |

### Example 3

TABLE 3

| SN | Ingredients | Mg/tablet |
|---|---|---|
| 1 | Trimetazidine | 35.000 |
| 2 | Microcrystalline cellulose | 53.000 |
| 3 | Polyox WSR coagulant LEO | 110.00 |
| 4 | Purified water | q.s. |

Coating

| | | |
|---|---|---|
| 5 | Hydroxypropyl methylcellulose | 15.525 |
| 6 | Ethyl cellulose | 7.475 |
| 7 | Titanium dioxide | 2.970 |
| 8 | Red iron oxide | 0.030 |
| 9 | Isopropyl alcohol | q.s. |
| 10 | Methylene chloride | q.s. |

| | |
|---|---|
| Final weight of the tablet | 196.000 |

### Procedure

1. Trimetazidine was blended with microcrystalline cellulose to form a mixture.
2. The mixture in step 1 was granulated with purified water to form granules.
3. The granules were dried and milled.
4. The milled granules were mixed with polyethylene oxide to form a blend.
5. The blend was compressed into suitable sized tablets.
6. Hydroxypropyl methylcellulose, ethyl cellulose, titanium dioxide and Red iron oxide were suspended in isopropyl alcohol and methylene chloride to form a coating suspension.
7. The tablets in step 5 were coated with coating suspension formed in step 6.

TABLE 4
In-vitro release pattern of modified release pharmaceutical
composition comprising Trimetazidine as per Example 3 in USP I
apparatus in 900 ml of phosphate buffer 6.8 pH at 100 RPM at a
temperature of 37° C. ± 0.5° C.

| Time (hr) | % Trimetazidine released |
|---|---|
| 0 | 0 |
| 1 | 26.88 |
| 2 | 57.05 |
| 3 | 66.00 |
| 4 | 76.47 |
| 5 | 82.81 |
| 6 | 88.62 |
| 7 | 91.57 |
| 8 | 93.49 |
| 9 | 95.92 |
| 10 | 96.79 |
| 11 | 96.11 |
| 12 | 95.85 |

### Example 4

TABLE 5

| SN | Ingredients | Mg/tablet |
|---|---|---|
| 1 | Trimetazidine | 35.000 |
| 2 | Microcrystalline cellulose | 55.000 |
| 3 | Polyox WSR 303 LEO | 110.000 |
| 4 | Purified water | q.s. |

Coating

| | | |
|---|---|---|
| 5 | Opadry pink | 4.000 |
| 6 | Isopropyl alcohol | q.s. |
| 7 | Methylene chloride | q.s. |

| | |
|---|---|
| Final weight of the tablet | 204.000 |

### Procedure

1. Trimetazidine was blended with microcrystalline cellulose to form a mixture.
2. The mixture in step 1 was granulated with purified water to form granules.
3. The granules were dried and milled.
4. The milled granules were mixed with polyethylene oxide to form a blend.
5. The blend was compressed into suitable sized tablets.
6. Opadry pink was suspended in isopropyl alcohol and methylene chloride to form a coating suspension.
7. The tablets in step 5 were coated with coating suspension formed in step 6.

TABLE 6
In-vitro release pattern of modified release pharmaceutical
composition comprising Trimetazidine as per Example 3 in USP I
apparatus in 900 ml of phosphate buffer 6.8 pH at 100 RPM
at a temperature of 37° C. ± 0.5° C.

| Time (hr) | % Trimetazidine released |
|---|---|
| 0 | 0.00 |
| 1 | 40.92 |
| 2 | 59.94 |
| 3 | 73.77 |
| 4 | 82.66 |
| 5 | 88.49 |
| 6 | 91.95 |
| 7 | 94.50 |
| 8 | 100.01 |
| 9 | 101.50 |
| 10 | 102.81 |
| 11 | 103.34 |
| 12 | 104.46 |

Certain modification and improvements of the disclosed invention will occur to those skilled in the art without departing from the scope of invention, which is limited only by the appended claims.

We claim:

1. A modified release solid pharmaceutical composition comprising Trimetazidine and polyethylene oxide, wherein the composition does not include any lubricant.

2. The solid pharmaceutical composition of claim 1, wherein the composition is in the form of one or more of tablet, capsule, powder, disc, caplet, granules, pellets, granules in capsule, minitablets, minitablets in capsule, pellets in capsule or sachet.

3. The solid pharmaceutical composition of claim 1, wherein the composition is in the form of tablet.

4. The solid pharmaceutical composition of claim 1, wherein the composition is in the form of capsule.

5. The solid pharmaceutical composition of claim 1, wherein the polyethylene oxide is having a viscosity in the range of 5,000 and 10,000 in 1% solution.

6. The solid pharmaceutical composition of claim 1, wherein the polyethylene oxide is present within 30% to 60% of the total weight of the composition.

7. The solid pharmaceutical composition of claim 1, wherein the composition comprises one or more of pharmaceutically acceptable inert excipients.

8. The solid pharmaceutical composition of claim 7, wherein the pharmaceutically acceptable inert excipients comprise one or more of fillers, binders and disintegrant.

9. The solid pharmaceutical composition of claim 8, wherein the fillers comprise one or more of microcrystalline cellulose, calcium phosphate, calcium sulfate, kaolin, starch, powdered sugar.

10. The solid pharmaceutical composition of claim 8, wherein the binders comprise one or more of povidone, starch, gums, hydroxypropylmethyl cellulose.

11. The solid pharmaceutical composition of claim 8, wherein the disintegrants comprise one or more of starch, croscarmellose sodium, crospovidone, sodium starch glycolate.

12. The solid pharmaceutical composition of claim 1, wherein the composition comprises a coating.

13. The solid pharmaceutical composition of claim 12, wherein the coating is functional coating.

14. The solid pharmaceutical composition of claim 13, wherein the coating comprises modified release polymers.

15. The solid pharmaceutical composition of claim 14, wherein the modified release polymer comprise one or more of water soluble polymer and water insoluble polymer.

16. The solid pharmaceutical composition of claim 14, wherein the modified release polymers comprise a combination of water soluble polymer and water insoluble polymer.

17. The solid pharmaceutical composition of claim 16, wherein the ratio of water soluble polymer and water insoluble polymer is from 1:5 to 5:1.

18. The solid pharmaceutical composition of claim 12, wherein the coating further comprises pharmaceutically acceptable inert excipients.

19. A process for the preparation modified release solid pharmaceutical composition comprising Trimetazidine and polyethylene oxide, wherein the composition does not include any lubricant, the process comprising the steps of:
(a) granulating Trimetazidine optionally with pharmaceutically acceptable inert excipients to form granules;
(b) drying the granules;
(c) mixing the dried granules with polyethylene oxide to form a blend; and
(d) compressing the blend into suitable sized tablet;
(e) optionally coating the tablet.

20. The process of claim 19, wherein the process comprises wet granulation process.
